(19) United States
(12) Patent Application Publication
(10) Pub. No.: US 2002/0015943 A1
(43) Pub. Date: Feb. 7, 2002

(54) Assays, methods and means relating to the modulation of levels of nuclear beta-catenin

(76) Inventor: Mariann Bienz, Lt. Shelford (GB)

Correspondence Address: Herbert B. Keil, KEIL & WEINKAUF, 1101 Connecticut Ave., N.W., Washington, DC, 20036, US

(21) Appl. No.: 09/915,307

(22) Filed: Jul. 27, 2001

Related U.S. Application Data
(60) Provisional application No. 60/221,892, filed on Jul. 31, 2000.

(51) Int. Cl. C12Q 1/00; G01N 33/574

(52) U.S. Cl. 435/4; 435/7.23

(57) ABSTRACT

Transcriptional changes mediated by high nuclear concentrations of β-catenin are known to be involved in the early stages of tumourigenesis. The present invention relates to the discovery that truncations of the tumour suppressor Adenomatous polyposis coli (APC) which are found in cancer cells cause high levels of nuclear β-catenin to accumulate by ‘trapping’ β-catenin within the nucleus. The high levels of ‘trapped’ nuclear β-catenin then affect transcription within the cell, Assays, methods and means are provided for modulating the interaction between modified APC and β-catenin, thereby lowering the nuclear concentration of β-catenin.

### FIELD OF INVENTION

This invention relates to the modulation of processes involved in the early stages of tumourigenesis, especially in colorectal cancers. Transcriptional changes mediated by high nuclear concentrations of β-catenin are known to be involved in these processes and this invention particularly relates to assays, methods and means of lowering levels of nuclear β-catenin.

### BACKGROUND OF INVENTION

Familial adenomatous polyposis is an inherited syndrome affecting about 1 in 700 individuals that inevitably causes colorectal cancer at around the fourth decade of life. FAP is caused by a mutation of the tumour suppressor Adenomatous polyposis coli (APC), which is also mutated in more than 80% of colorectal tumours (Kinzler, K. W. & Vogelstein, B. Cell 87, 159-170 (1996)).

Nearly all APC mutations are truncations, many of which terminate in the mutation cluster region (MCR) which is located in the central portion of the protein (Nagase, H. & Nakamura, Y. Hum. mutat, 2, 425-434 (1993), Miyaki, M. et al. Cancer Res. 54, 3011-3020 (1994) and Lamlum, H. et al. Nat Med 5, 1071-1075 (1999)). APC mutation is found in the smallest detectable adenomas and is thus the earliest known event in colorectal tumourigenesis APC truncation mutations have also been found in 17% of all breast cancers.

In normal cells, APC binds to cytosolic β-catenin, which is an effector of the Wnt signalling pathway. APC promotes the destabilisation of β-catenin by binding to the Axin complex which earmarks β-catenin for degradation by the proteasome pathway (Peifer, M. & Polakis, P. Science 287, 1606-1609 (2000)). APC has a regulatory role in this process (Behrens, J. et al. Science 280, 596-599 (1998) and Hart, M. J. et al. Curr Biol 8, 573-581 (1998)) which is poorly understood.

In APC mutant cancer cells, β-catenin is stabilised and accumulates in the cytoplasm, (Munemitsu, S. et al. Proc Natl Acad Sci USA 92, 3046-3050 (1995) and Morin, P. J. et al. Science 275, 1787-1790 (1997)) from where it translocates into the nucleus to serve as a transcriptional co-activator of TCF (T cell factor) (Korinek, V. et al. Science 275, 1784-1787 (1997)) and other tumour promoting genes. The transcriptional activity of β-catenin is critical for tumour development.

### SUMMARY OF INVENTION

The present inventors have shown that APC contains highly conserved nuclear export signals (NES) 3′ adjacent to the MCR which enable it to exit from the nucleus. This ability is lost in APC mutant cancer cells, and the work described herein shows that β-catenin accumulates in the nucleus as a result. The ability of APC to exit from the nucleus and thereby reduce the nuclear concentration of β-catenin, appears to be critical for its tumour suppressor function.

The present invention therefore relates to the unexpected discovery that the APC truncations found in cancer cells may ‘trap’ β-catenin in the nucleus. The trapped nuclear β-catenin then affects transcription.

One aspect of the present invention provides an assay method or a method of screening for an agent which decreases the amount of nuclear β-catenin in a cell, the method comprising;

contacting a modified APC polypeptide which binds β-catenin and has a reduced nuclear export activity, β-catenin polypeptide and a test compound; and,

determining binding of the modified APC polypeptide and the β-catenin polypeptide.

A method may be carried out under conditions in which the β-catenin polypeptide binds to the modified APC polypeptide in the absence of test compound.

A suitable modified APC polypeptide may have a C-terminus between amino acids 1263 and 1506 of the APC sequence (Acc No: P25054). Such a polypeptide may have an N-terminus at amino acid 1 of the published sequence.

The ability of the test compound to modulate binding may be determined by determining the binding of the β-catenin polypeptide and the APC polypeptide in the presence and absence of test compound. A difference in the amount of binding in the presence and absence of test compound being indicative of the test compound being a modulator of said binding interaction.

An assay method or method of screening as described herein may therefore include;

contacting a modified APC polypeptide which has a reduced nuclear export activity and which binds β-catenin, and a β-catenin polypeptide in the presence and absence of a test compound; and,

determining binding of said modified APC polypeptide and said β-catenin polypeptide

a difference in said binding in the presence relative to the absence of said test compound being indicative of said test compound being an agent which decreases the amount of nuclear β-catenin in a cell.

A modified APC polypeptide suitable for use in the methods described herein retains the ability to bind β-catenin but has a reduced, diminished, decreased or abolished nuclear export activity or function i.e. it is exported from the cell nucleus in reduced amounts or, more preferably is not exported from the nucleus of the cell at all, Such a modified, variant or mutant APC polypeptide may lack nuclear export signals.

Preferred modified APC polypeptides are truncated APC polypeptides expressed in tumour cells, particularly colorectal tumour cells. Examples include APC polypeptides with C terminal truncations as shown in FIG. 2. Suitable truncated APC polypeptides may have an N terminus at amino acid 1 and a C-terminus between amino acids 1263 and 1506 of the APC sequence (Acc No: P25054).

Particularly preferred is the truncated APC polypeptide expressed in the SW480 cell line which has a C terminal at amino acid 1338 of the published APC sequence (Acc No: P25054).

It will be understood that the precise C and N termini of a modified APC polypeptide as described herein are not crucial, as long as the modified APC polypeptide retains the ability to bind β-catenin and has decreased, or abolished nuclear export function. The termini may therefore be varied by one of skill in the art, for example by adding or deleting one or more, for example 2, 3, 4 or 5 amino acids from the N and/or C terminus of a modified APC polypeptide as described herein.

“β-catenin polypeptide” may be a polypeptide which has the published amino acid sequence of β-catenin (Acc No: X 87833) and which has the ability to bind APC.

“Full length APC polypeptide” is a polypeptide which has full nuclear export activity i.e. it is exported from the nucleus and binds β-catenin. Preferably, the polypeptide has the complete wild-type APC amino acid sequence (Acc No: P25054) which comprises NESs in the 20R3 and 20R4 repeats and is expressed in non-cancerous cells.

“Modified APC polypeptide” is a polypeptide which binds β-catenin but has a reduced, diminished, decreased or abolished nuclear export function i.e. it is not exported from the nucleus of a cell or is exported at decreased levels relative to the wild type APC protein (Acc No: P25054). A suitable modified APC polypeptide may be a fragment or truncated form of the full length APC sequence as described herein.

Instead of using wild-type, the β-catenin polypeptide, APC polypeptide and full-length APC polypeptide employed in various aspects and embodiments of the present invention may include an amino acid sequence which differs by one or more amino acid residues from the wild-type amino acid sequence, by one or more of addition, insertion, deletion and substitution of one or more amino acids, for example at the N and/or C termini as described above. Thus, variants, derivatives, alleles, mutants and homologues, e.g. from other organisms, are included.

Preferably, the amino acid sequence of the APC polypeptide, full length APC polypeptide or β-catenin polypeptide shares homology with the corresponding sequence of the published APC or β-catenin sequences (Acc No: P25054, Acc No: X 87838) as the case may be, preferably at least about 70%, or 80% homology, or at least about 90% or 95% homology.

As is well-understood, homology at the amino acid level is generally in terms of amino acid similarity or identity. Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Similarity may be as defined and determined by the TBLASTN program, of Altschul et a1. (1990) J. Mol. Biol. 215: 403-10, which is in standard use in the art. Homology may be over the full-length of the relevant polypeptide or may more preferably be over a contiguous sequence of about 15, 20, 25, 30, 40, 50 or more amino acids, compared with the relevant wild-type amino acid sequence. Preferred sequences of “APC polypeptide”, “full length APC polypeptide” and “β-catenin polypeptide” may share at least about 70%, 80%, 85%, 88%, 90% or 95% identity with the corresponding sequence in the respective published sequences (Acc No: P25054, Acc No: X 87838).

Thus, fragments, mutants, variants, alleles, derivatives homologues and analogues may be used, within the meaning of “truncated APC polypeptide”, “full length APC polypeptide” or “β-catenin polypeptide”. Suitable molecules retain the biological activity of binding to an APC polypeptide or binding to an β-catenin polypeptide, as the case may be,

As stated above, it is not always necessary to use the entire APC or β-catenin proteins for assays of the invention. Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers, Small fragments (e.g. up to about 20 or 30 amino acids) may also be generated using peptide synthesis methods which are well known in the art.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

The ability of suitable fragments of the full length APC sequence to bind to β-catenin (or fragment thereof), or suitable fragments of β-catenin to bind to APC (or fragment thereof), may be tested using routine procedures such as those illustrated in the accompanying examples.

A “fragment” of the polypeptide means a stretch of amino acid residues of at least about five to seven contiguous amino acids, often at least about seven to nine contiguous amino acids, typically at least about nine to 13 contiguous amino acids and, more preferably, at least about 20 to 30 or more contiguous amino acids. Fragments of a polypeptide may include antigenic determinants or epitopes useful for raising antibodies. Alanine scans are commonly used to find and refine peptide motifs within polypeptides, this involving the systematic replacement of each residue in turn with the amino acid alanine, followed by an assessment of biological activity.

A “derivative” of a polypeptide or a fragment thereof may include a polypeptide modified by varying the amino acid sequence of the protein, e.g. by manipulation of the nucleic acid encoding the protein or by altering the protein itself. Such derivatives of the natural amino acid sequence may involve one or more of insertion, addition, deletion or substitution of one or more amino acids, as discussed.

Although the relevant polypeptide may be provided in free form, it may also be used in the form of a fusion protein linked to a marker, label or reporter protein. For example, in a preferred embodiment of the invention, the APC or β-catenin polypeptide may be fused to a heterologous DNA binding domain such as that of the yeast transcription factor GAL 4. The GAL 4 transcription factor includes two functional domains. These domains are the DNA binding domain (DBD) and the transcriptional activation domain (TAD). By fusing APC polypeptide or β-catenin polypeptide to one of those domains and the respective counterpart, i.e. β-catenin polypeptide or APC polypeptide, to the other domain, a functional GAL 4 transcription factor is restored only when two proteins of interest interact. Thus, interaction of the proteins may be measured by the use of a reporter gene probably linked to a GAL 4 DNA binding site which is capable of activating transcription of said reporter gene. This assay format is described by Fields and Song, 1989, Nature 340; 245-246. This type of assay format can be used in both mammalian cells and in yeast.

The precise format of the assay of the invention may be varied by those of skill in the art using routine skill and knowledge. For example, the interaction between the polypeptides may be studied in vitro by labelling one with a detectable label and bringing it into contact with the other which has been immobilised on a solid support. Suitable detectable labels include ³⁵S-methionine which may be incorporated into recombinantly produced peptides and polypeptides. Recombinantly produced peptides and polypeptides may also be expressed as a fusion protein containing an epitope which can be labelled with an antibody.

The protein which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Fusion proteins may be generated that incorporate six histidine residues at either the N-terminus or C-terminus of the recombinant protein. Such a histidine tag may be used for purification of the protein by using commercially available columns which contain a metal ion, either nickel or cobalt (Clontech, Palo Alto, Calif., USA). These tags also serve for detecting the protein using commercially available monoclonal antibodies directed against the six histidine residues (Clontech, Palo Alto, Calif., USA).

An assay according to the present invention may also take the form of an in vivo assay. The in vivo assay may be performed in a cell line, preferably a mammalian cell line in which the relevant polypeptides are expressed from one or more vectors introduced into the cell. Alternatively, the assay may be performed using a cell line which endogenously expresses truncated APC and β-catenin, for example a colorectal tumour cell line such as SW480. The interactions of truncated APC polypeptide, β-catenin polypeptide and the test compound may thereby be determined in the nucleus of a cell.

The test compound, for example a peptide, may be added to culture medium containing the appropriate cells, which then take up the test compound.

Cells suitable for use in assays as described herein include cancer cells, preferably colorectal cancer cells, for example SW480. The APC mutation in SW480 cells gives rise to aggressive colorectal tumours This may indicate a particularly strong truncated APC and β-catenin interaction in the nucleus of these cells. SW480 cells are therefore especially preferred in assays of the present invention.

Binding of truncated APC and β-catenin in an in vivo assay may be determined by determining the concentration of nuclear β-catenin, for example by measuring the transcriptional activity of β-catenin, An assay may be performed in cells which contain a reporter gene such as luciferase or GFP linked to a TCF binding site and a minimal promoter (for example TOPFLASH). The signal from the reporter gene is related to the level of nuclear β-catenin. Compounds which reduce or inhibit the binding of nuclear β-catenin to truncated APC cause a reduction in the concentration of nuclear β-catenin and therefore a reduction in reporter signal. Other methods of determining β-catenin concentration, such as antibody staining are well known to those of skill in the art.

To target the activity of the agent to particular cells and reduce unwanted side effects, it is desirable that the agent inhibits the binding of the truncated APC to a greater extent than the binding of the full length APC. A agent obtained by an assay of the present invention therefore preferably modulates, disrupts, interferes with or inhibits the binding of β-catenin to modifiedAPC preferentially over the binding of β-catenin to full length APC.

A further aspect of the present invention therefore provides for a method of screening for agents selective for the modifiedAPC interaction.

A method of screening may therefore include the steps; contacting a truncated AFC polypeptide having an N terminus at amino acid 1 and a C-terminus between amino acids 1263 and 1506 of the APC sequence (P25054), β-catenin polypeptide and a test compound; and, determining binding of the modified APC polypeptide and the β-catenin polypeptide contacting a full length APC polypeptide, the β-catenin polypeptide and the test compound; and, determining relative binding of the full length APC polypeptide to β-catenin polypeptide compared with the binding of the modified APC polypetide to β-catenin polypeptide,

Relative binding may be determined by determining the binding of the full length APC polypeptide to β-catenin polypeptide in the presence of test compound and comparing this binding with the binding of the modified APC polypeptide to β-catenin polypeptide in the presence of test compound. Relative binding may be expressed as a ratio, fraction, multiple or percentage of the modified APC polypeptide binding.

The binding of the modified APC polypeptide and β-catenin polypeptide may be determined in the nucleus of a cell and binding of full length APC polypeptide and β-catenin polypeptide may be determined in the cytoplasm of a cell.

High levels of β-catenin occur in the nucleus as a result of binding to modified APC polypeptide which retains β-catenin binding activity but has lost nuclear export function, Such trapping of β-catenin in the nucleus may be prevented by reducing the ability of modified APC polypeptide to enter the nucleus. Nuclear entry therefore provides an additional target for the modulation of the concentration of nuclear β-catenin.

A further aspect of the present invention therefore provides an assay method or method of screening for an agent which reduces nuclear β-catenin in a cell comprising;

introducing a test compound to the cytoplasm of a cell, wherein said cytoplasm contains a modified APC polypeptide having an N terminus at amino acid 1 and a C-terminus between amino acids 1263 and 1506 of the APC sequence (Acc No: P25054); and,

determining the level, amount or concentration of modified APC polypeptide in the nucleus of said cell.

The concentration of modified APC polypeptide in the nucleus of said cell may be determined in the presence and absence of said test compound. A decrease in the concentration of modified APC polypeptide in the nucleus of said cell in the presence relative to the absence of said test compound is indicative of said test compound being an agent which reduces nuclear β-catenin in a cell.

An agent may bind to the modified APC polypeptide and prevent passage into the nucleus, for example through binding to Armadillo Repeat Domain (ARD). Alternatively an agent may interact with receptors for modified APC on the surface of the nuclear membrane, particularly receptors which recognise ARD.

Modified APC polypeptide may be expressed endogenously by the cell, or may be exogenous, e.g. expressed on an expression vector.

A skilled person is aware of the need for controls will. perform suitable control experiments as and where necessary in carrying out the assays of the present invention.

A further aspect of the present invention provides, following obtaining an agent employing a method as described herein, providing the agent to a cell to reduce nuclear β-catenin in the cell.

Such a cell may be a cancer cell, in particular a colorectal cancer cell. The cell may be a cultured cell (in vitro) or may be a cell within the body of a patient (in vivo). The agent may be provided for a therapeutic purpose, for example, the alleviation or amelioration of a condition such as cancer.

Methods as described herein may include determining ability of the test compound to reduce nuclear β-catenin in a cell.

Combinatorial library technology (Schultz, JS (1996) Biotechnol. Prog. 12:729-742) provides an efficient way of testing a potentially vast number of different compounds for ability to modulate activity of a polypeptide. Prior to or as well as being screened for modulation of activity, test compounds may be screened for ability to interact with the polypeptide, e.g. in a yeast two-hybrid system (which requires that both the polypeptide and the test compound can be expressed in yeast from encoding nucleic acid). This may be used as a coarse screen prior to testing a compound for actual ability to modulate activity of the polypeptide.

The amount of test substance or compound which may be added to an assay of the invention will normally be determined by trial and error depending upon the type of compound used. Typically, from about 0.01 to 100 nM concentrations of putative inhibitor compound may be used, for example from 0.1 to 10 nM. Greater concentrations may be used when a peptide is the test compound.

Compounds which may be used may be natural or synthetic chemical compounds used in drug screening programmes. Extracts of plants which contain several characterised or uncharacterised components may also be used. A further class of putative inhibitor compounds can be derived from the APC polypeptide and/or the β-catenin polypeptide which binds to it, Peptide fragments of from 5 to 40 amino acids, for example from 6 to 10 amino acids from the region of the relevant polypeptide responsible for interaction, may be tested for their ability to disrupt such interaction. Preferred peptide fragments may comprise or consist of one or more 20 amino acid repeat β-catenin binding motifs derived from the full length APC sequence (see FIG. 1).

Antibodies directed to the site of interaction in either APC or β-catenin, for example the 20R motif of APC, form a further class of putative inhibitor compounds. Candidate inhibitor antibodies may be characterised and their binding regions determined to provide single chain antibodies and fragments thereof which are responsible for disrupting the interaction. Peptide, polypeptide and antibody inhibitors may be expressed in a cell and targeted to the nucleus using a nuclear localisation signal (NLS). Alternatively, test compounds may be added to cells in culture medium, so that the cells take up the test compound.

Other candidate inhibitor compounds may be based on modelling the 3-dimensional structure of a polypeptide or peptide fragment and using rational drug design to provide potential inhibitor compounds with particular molecular shape, size and charge characteristics.

A further aspect of the present invention provides an agent, compound or substance which is obtained by an assay method as described herein and which modulates or affects nuclear β-catenin levels. Such an agent, compound or substance may inhibit the binding of nuclear β-catenin and modified APC polypeptide or inhibit the importation of modified APC into the cell nucleus.

Following identification of a agent, compound or substance which modulates or affects nuclear β-catenin levels using an assay as described herein, the compound may be investigated further. An agent, compound or substance may be isolated and/or purified, manufactured and/or used in preparation, i.e. manufacture or formulation, of a composition such as a medicament, pharmaceutical composition or drug. These may be administered to individuals.

Thus, the present invention extends in various aspects not only to a compound identified using an assay as described herein as an agent which is a modulator of nuclear β-catenin levels, in accordance with what is disclosed herein, but also a pharmaceutical composition, medicament, drug or other composition comprising such a compound, a method comprising administration of such a composition to a patient, e.g. for reducing nuclear β-catenin levels for instance in treatment (which may include preventative treatment) of a cancer such as colorectal cancer, for example FAP, use of such a compound in manufacture of a composition for administration, e.g. for reducing nuclear β-catenin levels for instance in treatment (which may include preventative treatment) of a cancer such as colorectal cancer, for example FAP, and a method of making a pharmaceutical composition comprising admixing such a compound with a pharmaceutically acceptable excipient, vehicle or carrier, and optionally other ingredients.

A further aspect of the present invention provides a method of treatment of cancer, preferably colorectal cancer such as FAP, comprising administration of an agent as described herein to a individual in need thereof.

A compound identified as a modulator of nuclear β-catenin levels using an assay of the present may be peptide or non-peptide in nature. Non-peptide “small molecules” are often preferred for many in vivo pharmaceutical uses. Accordingly, a mimetic or mimick of the compound (particularly if a peptide) may be designed for pharmaceutical use. The designing of mimetics to a known pharmaceutically active compound is a known approach to the development of pharmaceuticals based on a “lead” compound, This might be desirable where the active compound is difficult or expensive to synthesise or where it is unsuitable for a particular method of administration, e.g. peptides may not be well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or sub-cutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Targeting may also be employed to direct the active agent to the nucleus of a cell, for example by coupling to a nuclear localisation signal.

Instead of administering an agent directly, it may be produced in target cells by expression from an encoding gene introduced into the cells, e.g. in a viral vector (see below). The vector may be targeted to the specific cells to be treated, or it may contain regulatory elements which are switched on more or less selectively by the target cells. Viral vectors may be targeted using specific binding molecules, such as a sugar, glycolipid or protein such as an antibody or binding fragment thereof Nucleic acid may be targeted by means of linkage to a protein ligand (such as an antibody or binding fragment thereof) via poly-lysine, with the ligand being specific for a receptor present on the surface of the target cells.

An agent may be administered in a precursor form, for conversion to an active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WC 90/07936).

Aspects of the present invention will now be illustrated with reference to the accompanying figures described already above and experimental exemplification, by way of example and not limitation Further aspects and embodiments will be apparent to those of ordinary skill in the art. All documents mentioned in this specification are hereby incorporated herein by reference.

### BRIEF DESCRIPTION OF THE FIGURES

FIGS. 1 and 2 show maps of APC proteins and positions of NESs relative to the MCR.

FIG. 1 shows (Top) E-APC with conserved domains (black bars, 20Rs, hollow bar 15Rs; grey bar, Axin binding site; black block, ARD), and maps of ARDcore and Cterm2 (Cterm1 is the same as Cterm2, but terminates at codon 908, 5′ to the Axin binding site). Grey arrows mark functional NESs. An untested NES candidate is indicated by black arrow, a non-functional NES candidate by arrowhead. Bottom, sequences of 20R3 and 20R4 of E-APC (above) and human APC (underneath), with conserved NES residues marked by dots.

FIG. 2 shows Human AFC with conserved domains, and functional and untested NESs marked as in (a). The MCR is bracketed, and expanded below to show the codon positions of 315 somatic truncation mutations from colorectal tumours (Lamlum H. et al (1999) Nat Med 5 1071-1075) (dots; double-bars indicate 10 additional mutations at each hot-spot; see also APC Mutation Database, http.//perso.curie.fr/Tierry.Soussi/APC.html). Note abrupt 3′ border of mutations immediately upstream of the 20R3 NES.

FIG. 3 shows complementation tests in APC mutant cancer cells. Transcriptional read-outs of nuclear β-catenin in SW480 cells transfected with GFP (Mock), HC, HCala, HCala1 or HCala2 (with one or two 20R NESs retained, respectively; see text). Grey columns, TOPFLASH; black columns, FOPFLASH. HCala is significantly less active than HC (or HCala2) in reducing transcriptional activity of nuclear β-catenin.

### DETAILED DESCRIPTION OF THE INVENTION

Methods

Constructs and Luciferase Assays

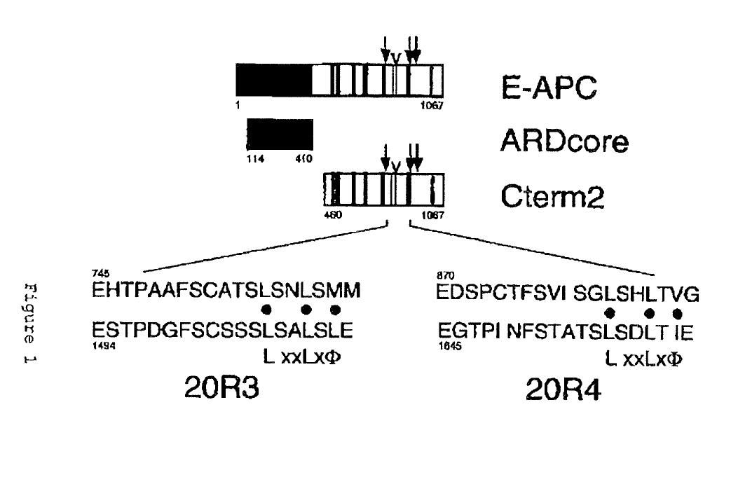

All constructs were inserted into pEGFP-C2 (Clontech), producing N-terminally tagged GFP fusions. Each construct (except HC and HCala, see below) also contains a triple hemagglutinin (HA) tag inserted between GFP and APC coding sequence. In coreNES, the following NES-encoding sequences were inserted between the HA tag and the ARDcore (first of a pair human, second Drosophila, bold NES residues were substituted to alanine in coreNESala; in coreNEScon, underlined conserved non-NES residues were substituted by alanine): 203, ESTPDGFSCSSSLSALSLDEP, EHTPAAFSCATSLSNLSMMDD; 20R4, EGTPINFSTATSLSDLTIESP, EDSPCTFSVISGLSHLTVGSA; 20R7, EDTPVCFSRNSSLSSLSIDSE. coreNESmin from Drosophila 20R4 contains ISGLSHLTVGSA (bold residues substituted by alanine in the mutant version). We also fused the putative NES from Drosophila pseudoR20 (FIG. 1, arrowhead) to ARDcore, but this NES candidate (EDTTAVLSKAPSNSCLSILSIPND) was non-functional. For the complementation assays in SW480 cells, a central fragment from human APC (codons 1379-2080) was N-terminally tagged with GFP (HC, see above). The above described alanine substitutions of 20R-linked NESs were introduced into HC, and into GFP-E-APC, using the Quik-Change Site-Directed Mutagenesis Kit (Stratagene), HCala1 and HCala2 are partial mutants retaining one and two NESs, respectively (see text) All constructs were verified by sequencing.

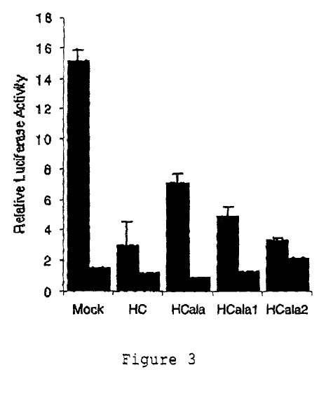

pTOPFLASH and pFOPFLASH were used for transcriptional read-out assays of nuclear β-catenin in SW480 cells (Korinek, V. et al. Science 275, 1784-1787 (1997)). pRL-CMV served as an internal control, and luciferase assays were performed with the Dual Luciferase™ Reporter Assay System (Promega). Relative luciferase activities (x100) were obtained by dividing TOP- or FOP-FLASH values by pRL-CMV values (FIG. 3). TOP-FLASH values reflect averages of 2-4 independent transfections, their standard deviations are given.

Tissue Culture, Fly Embryos and Immunofluorescence

Monkey COS cells were grown in DMEM medium (supplemented with 10% fetal calf serum), and transfected with FuGENE™ (Morin, P. J. et al. Science 275, 1787-1790 (1997)) Transfection Reagent (Roche). SW480 and HCT116 cells were grown in Leibovitz's L15 medium (with 10% fetal calf serum), and transfected with Lipofectamine (Life Technologies Inc.). 0.4 or 0.8 mg DNA was used per transfection of COS or SW480 cells (in 35 mm culture wells), respectively. Transfected cells were harvested for analysis after 24-48 hours. For staining, cells were either fixed with chilled methanol for 10 min. at −20° C. or fixed with 4% paraformaldehyde (freshly prepared, in phosphate buffered saline) for 30 min. Cells were then permeabilised with 0.1% Triton X-100 and blocked with bovine serum albumin for 15 min. each, and subsequently incubated at room temperature with primary and secondary antibody for 60 and 30 min, respectively. COS cells were treated with LMB (50 ng/ml)) for 1-2 hours 24 hours after transfection, SW480 cells for 2 hours 48 hours after transfection.

Drosophila embryos were fixed and stained as described (Yu, X. et al. Nature Cell Biol 3, 144-151 (1999)). For drug treatments, 0-6 hours old embryos were permeabilised in octane (Lantz, V. A. et al. Mech Dev 85, 111-122 (1999)) and subsequently incubated for 60 min. with 80 ng/ml LMB. Controls were octane-permeabilised and incubated in solvent alone (<1% ethanol).

The following antibodies were used: rabbit anti-E-APC (1:10′000) (Yu, X. et al. Nature Cell Biol 3, 144-151 (1999), rabbit anti-APC (1:700) (Näthke, I. S. et al. J Cell Biol 134, 165-179 (1996)), mouse anti-lamin (1:150), mouse anti-β-catenin (1:500; Transduction Laboratories), Alexa Red and Green (1:500; Molecular Probes). images were collected on an MRC 1024 confocal microscope.

Some heterogeneity was observed with the NES constructs, and with the SW480 complementation assays. Qualitative analysis was thus backed up by quantitative evaluation of 10-20 randomly chosen fields in which each individual healthy cell was scored. Ratios of nuclear to cytoplasmic fluorescence levels were determined.

Expression levels of GFP fusions were checked by western blotting, essentially as described (Shih, I. M. et al. Cancer Res 60, 1671-1676 (2000)), The following antibody dilutions were used: anti-APC (see above), 1:2000; rat anti-HA (clone 3F10, Roche), 1:700; anti-a-tubulin (Sigma T9026), 1:500.

In initial experiments, COS cells were transfected with GFP-tagged constructs as described, with or without LMB. Cterm1 was found to be excluded from nuclei at least as efficiently as Cterm2. This exclusion, like that of Cterm2, was blocked by LMB. 4-5 hours old Drosophila embryos were stained with anti-E-APC which outlined the apical cellular junctions, and anti-lamin which outlined the nuclear envelopes E-APC was found to accumulate in the nuclei after LMB treatment, COS cells were transfected with coreNES constructs; ARDcore; coreNES from E-APC 20R4; coreNESala from E-APC 20R4; coreNES from human APC 20R3, and coreNESala from human APC 20R3. All coreNES constructs described in the text were found to behave in essentially the same way.

APC proteins are found in multiple sub-cellular compartments of mammalian and Drosophila cells including cytoplasm, nucleus and adhesive cadherin/catenin junctions (Yu, X. et al. Nature Cell Biol 3, 144-151 (1999), McCartney, B. M. et al J Cell Biol 146, 1303-1318 (1999) and Neufeld, K. L. & White, R. L. Proc Natl Acad Sci USA 94, 3034-3039 (1991)). To identify the targetting domains for these compartments, we tagged various fragments of the ubiquitously expressed Drosophila APC, called E-APC/dAPC2, with green fluorescent protein (GFP) and expressed these in transgenic fly embryos and in monkey COS cells.

The subcellular distribution of GFP-E-APC was indistinguishable from that of endogenous E-APC in embryos (Yu, X. et al. Nature Cell Biol 3, 144-151 (1999)). In COS cells transfected with GFP-E-APC, we saw green fluorescence in the cytoplasm, somewhat concentrated at the plasma membrane but also some in the nucleus. Unexpectedly, an N-terminal fragment of E-APC (ARDcore) accumulated in the nucleus. Evidently, E-APC is capable of entering the nucleus by virtue of its N-terminus. We have not studied this further, but we note that this N-terminus spans the highly conserved Armadillo repeat domain (ARD).

In contrast, C-terminal fragments of E-APC (Cterm1 and 2; FIG. 1) were efficiently excluded from the nucleus, more so than the full-length protein. We tested our GFP constructs by treating transfected cells with leptomycin B (LMB), a highly specific drug that inhibits nuclear export by directly blocking the nuclear export receptor CRM1 (Fukuda, M. et al. Nature 390, 308-311 (1997), Fornerod, M. et al. Cell 90, 1051-1060 (1997) and Kudo, N. et al. Exp Cell Res 242, 540-547 (1998)). This resulted in even distribution of Cterm1 and 2 throughout cytoplasm and nucleus, Full-length E-APC also accumulated to some extent in the nucleus after LMB treatment (not shown). Importantly, endogenous E-APC is retained efficiently in nuclei of LMB-treated Drosophila embryos. These results indicated the presence of an NES in the C-terminus of E-APC.

We scanned through the C-terminal sequence of E-APC for matches to the leucine-rich NES consensus sequence (LxxLxF; F being L, I, M or V) We found two matches in intriguing positions, namely within the so-called 20 amino acid repeats 3 and 4 (20R3, 20R4) (FIG. 1, grey arrows). The 20Rs are highly conserved motifs (black bars in FIG. 1) which in human APC are known to bind β-catenin (Munemitsu, S, et al. Proc Natl Acad Sci USA 92, 3046-3050 (1995), Su, L. K. et al. Science 262, 1734-1737 (1993) and Rubinfeld, B. et al. Cancer Res 57, 4624-4630 (1997)). These putative NESs in 20R3 and 20R4 are conserved in all known APC proteins. Human APC contains an additional NES match in 20R7 (FIG., 2, grey arrow). Two further matches were found in both proteins (FIG. 1, black arrows, arrowhead). We tested the R20-linked NESs from E-APC and human APC, by fusing each individually to the ARDcore (coreNES). We also generated mutant versions which bear alanine substitutions of the conserved NES residues (coreNESala), as well as a control alanine mutant (coreNEScon) and a minimal NES construct (coreNESmin; see Methods),

We found that all coreNES fusions are efficiently excluded from nuclei of transfected COS cells. Similarly, cells transfected with coreNESmin and coreNEScon showed almost no green fluorescence in the nuclei. In contrast, green fluorescence from coreNESala mutants was evenly spread throughout cytoplasm and nuclei of transfected cells. These mutants were thus indistinguishable from ARDcore. LMB treatment abolishes the nuclear exclusion of all coreNES fusions, but neither affected the subcellular distribution of RRDcore nor that of any coreNESala mutant. The 20R-linked NESs from human and Drosophila APC therefore function as nuclear export signals.

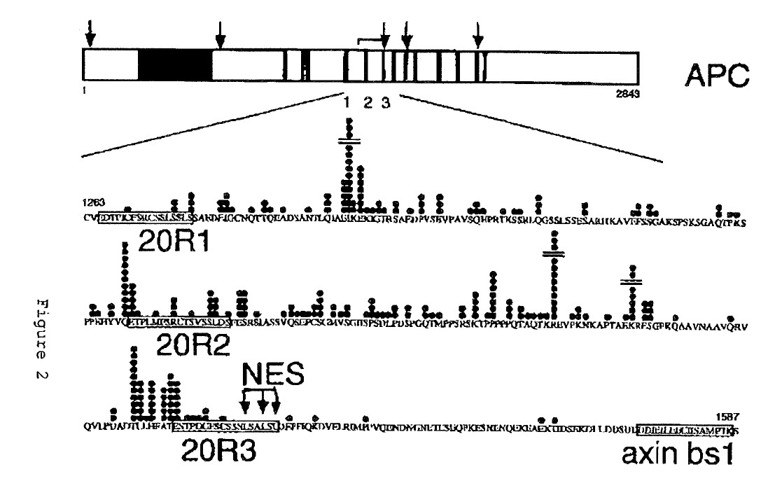

The 20R3 NES overlaps the MCR of human APC. We plotted the codon positions of 315 somatic mutations from colorectal tumours (Nagase, H. & Nakamura, Y. Hum. mutat. 2, 425-434 (1993), Miyaki, M. et al. Cancer Res. 54, 3011-3020 (1994) and Lamlum, H. et al. Nat Med 5, 1071-1075 (1999)) to reveal a fairly even spread throughout the MCR, with known hot-spots, up to an abrupt 3′ border immediately upstream of the 20R3 NES (codon 1506; FIG. 2). Only 5 mutations fell within the 72 codons between this border and the 5′ most Axin binding motif (codon 1570; FIG. 2) which is thought to be critical for the tumour suppressor function of APC (Smits, R. et al. Genes Dev 13, 1309-1321 (1999) and Shih, I. M. et al. Cancer Res 60, 1671-1676 (2000)). Of 573 somatic colorectal tumour mutations compiled in the APC database, only 16 (2.8%) fell 3′ to this border. Germ line mutations 3′ to codon 1465 usually lead to fewer polyps than those located more upstream, and those 3′ to 1578 are associated with attenuated polyposis (Lal, G. & Gallinger, S. Sem Surg. Onco. 18, 314-323 (2000)). The 3′ border that we observed in this analysis indicates a strong selection against the presence of the 20R-linked NESs in cancers. This provides indication that the ability of APC to exit from the nucleus is involved in its tumour suppressor function.

We examined the sub-cellular distribution of endogenous APC in HCT116 colon cancer cells which contain wild-type APC, and in SW480 cells whose resident modified APC lacks all 20R-linked NESs (Morin, P. J. et al. Science 275, 1787-1790 (1997) and Rowan, A. J. et al. Proc Natl Acad Sci USA 97, 3352-3357 (2000)), using an antiserum raised against a central fragment of human APC (Näthke, I. S. et al. J Cell Biol 134, 165-179 (1996)). This revealed that HCT116 cells contained largely cytoplasmic APC some of which was associated with the plasma membrane, but very little was seen in the nucleus. In contrast, in many SW480 cells, the modified APC was predominantly nuclear; in some cells, it was spread evenly throughout nucleus and cytoplasm. Evidently, this APC truncation had lost most of its nuclear export function, providing indication that its N-terminal NES matches did not significantly contribute to this function in the mutant protein. Interestingly, these sub-cellular distributions of APC were largely mirrored by β-catenin: in HCT116 cells, β-catenin was mostly associated with the plasma membrane, being barely detectable elsewhere, whereas in many SW480 cells, β-catenin was concentrated in nuclei. This provided further indication that the sub-cellular distribution of β-catenin is a consequence of that of APC.

We generated NES-less APC mutants by introducing all the above-described NESala substitutions into a GFP-tagged central fragment of human APC (HC) to generate HCala, and into full-length E-APC (E-APCala) since E-APC also reduced β-catenin in SW480 cells (Hamada, F. et al. Genes Cells 4, 465-474 (1999)). As expected, wild-type HC efficiently exited from nuclei, and strongly reduced nuclear β-catenin in transfected SW480 cells. We observed some variability of this effect, e.g. cells with low HC levels tended to retain cytoplasmic β-catenin, but we rarely saw a transfected cell whose β-catenin was higher in the nucleus than in the cytoplasm. LMB treatment caused nuclear retention of HC, and also attenuated the reduction of nuclear β-catenin by HC in that many HC-transfected cells had more β-catenin in the nuclei than in the cytoplasm. Similarly, HCala, typically retained in nuclei of transfected cells, was compromised in its ability to reduce their nuclear β-catenin levels. However, the cytoplasmic β-catenin levels on the whole were still reduced in LMB-treated and HCala-transfected cells, indicating that the cytoplasmic APC in these cases retained the ability to destabilise β-catenin (note that HCala most probably still binds β-catenin (Rubinfeld, B. et al. Cancer Res 57, 4624-4630 (1997)) and Axin (Behrens, J. et al. Science 280, 596-599 (1998) and Hart, M. J. et al. Curr Biol 8, 573-581 (1998)). Similar results were obtained with E-APCala which was significantly less active in reducing nuclear β-catenin than its wild-type counterpart.

To quantitate the activities of HC and HCala in reducing nuclear β-catenin, we used a transcriptional read-out based on a luciferase reporter linked to TCF binding sites (TOPFLASH; the FOPFLASH control contains mutant TCF binding sites) (Korinek, V. et al. Science 275, 1784-1787 (1997)). As expected, the high luciferase activity of mock-transfected SW480 cells was much reduced by HC, but less so by HCala (FIG. 2). A partially mutant HCala which retained the 20R4 and 20R7 NESs (HCala2) was nearly as active as wild-type HC in reducing luciferase activity, while a mutant retaining only the 20R7 NES (HCala1) was less active (FIG. 2). These results confirmed the functional importance of the 20R-linked NESs of APC in reducing the nuclear β-catenin in APC mutant cancer cells.

We have shown that APC proteins contain highly conserved and functional nuclear export signals. The close relationship between the ability of APC to exit from the nucleus and its tumour suppressor function is shown by three lines of evidence: the sharp 3′ border of APC truncation mutations, the nuclear accumulation of modified APC (lacking 20R-linked NESs) in APC mutant cancer cells, and the compromised ability of NES-less APC to reduce nuclear β-catenin in these cells. The nuclear export function of APC appears to be the 5′ most tumour suppressor function within the protein. APC's ability to bind Axin in order to destabilise β-catenin, also clearly critical for its tumour suppressor function (Smits, R. et al. Genes Dev 13, 1309-1321 (1999) and Shih, I. M. et al. Cancer Res 60, 1671-1676 (2000)), is encoded slightly further downstream, and additional functions may reside in its C-terminus (Peifer, M. & Polakis, P. Science 287, 1606-1609 (2000)).

The present investigation provides indication that APC shuttles β-catenin/Armadillo from the nucleus and cytoplasm to the junctional compartment where the Axin complex appears to be anchored (Bienz, M. Curr Opin Genet Dev 9, 595-603 (1999)). Our work demonstrates this shuttling function of APC since the subcellular distribution of β-catenin mirrors that of APC in wild-type and APC mutant cancer cells, The nuclear β-catenin in the cancer cells does not simply reflect the loss of APC-mediated export but also provides indication that β-catenin is positively trapped in the nuclei by the mutant APC. Nuclear trapping of β-catenin by modified APC provides a mechanistic explanation for the striking mutation pattern observed in colorectal tumours which reveals a strong selection for 20R1 to be retained in at least one of the two mutant APC alleles (Lamlum, H. et al. Nat Med 5, 1071-1075 (1999)).

1. An assay method for identifying an agent which decreases the amount of nuclear β-catenin in a cell, the method comprising;
contacting a modified APC polypeptide which binds β-catenin and has a reduced nuclear export activity with a β-catenin polypeptide in the presence and absence of a test compound, and,
determining binding of said modified APC polypeptide and said β-catenin polypeptide
a difference in said binding in the presence relative to the absence of said test compound being indicative of said test compound being an agent which reduces nuclear β-catenin in a cell.

2. A method according to claim 1 wherein said modified APC polypeptide and said β-catenin polypeptide are in the nucleus of a cell.

3. A method according to claim 2 further comprising the steps;
contacting a full-length APC polypeptide, a β-catenin polypeptide in the presence and absence of said test compound; and,
determining the binding of the β-catenin polypeptide to full-length APC polypeptide compared to the modified APC polypeptide in the presence of said test compound.

4. A method according to claim 3 wherein said full-length APC polypeptide and said β-catenin polypeptide are contacted in the cytoplasm of said cell.

5. An assay method for an agent which decreases the amount of nuclear β-catenin in a cell comprising;
introducing a test compound to the cytoplasm of a cell, wherein said cytoplasm contains a modified APC polypeptide which binds β-catenin and has a reduced nuclear export activity, with a β-catenin polypeptide; and,
determining the amount of modified APC polypeptide in the nucleus of said cell,
a decrease in the amount of modified APC polypeptide in the nucleus of said cell being indicative of said test compound being an agent which decreases the amount of nuclear β-catenin in a cell.

5. A method according to claim 1 or claim 4 wherein the cell is a cancer cell.

6. A method according to claim 5 wherein the cell is a colorectal cancer cell.

7. A method according to claim 1 or claim 4 comprising identifying the test compound as an agent which decreases the amount of nuclear β-catenin in a cell.

8. A method according to claim 7 comprising formulating the agent into a composition including a pharmaceutically acceptable excipient.

9. A method comprising, following obtaining an agent employing a method of claim 1; providing the agent to a cell to reduce the nuclear concentration of β-catenin in said cell.

10. A method according to claim 9 wherein said cell is a cell within the body of a patient.
